(19) United States
(12) Patent Application Publication
(10) Pub. No.: US 2014/0324142 A1
(43) Pub. Date: Oct. 30, 2014

(54) ACUTE MYOCARDIAL INFARCTION TREATMENT BY ELECTRICAL STIMULATION OF THE THORACIC AORTA

(71) Applicant: ENOPACE BIOMEDICAL LTD., Caesarea (IL)

(72) Inventors: Amir Dagan, Kibbutz Megiddo (IL); Offer Glasberg, Zichron Ya'akov (IL); Yotam Reisner, Kiryat Tivon (IL); Gal Ariav, Givaat Ada (IL); Nitai Hanani, Haifa (IL)

(21) Appl. No.: 14/356,829

(22) PCT Filed: Nov. 8, 2012
(86) PCT No.: PCT/IL12/50452

§ 371 Date: May 7, 2014

Related U.S. Application Data
(60) Provisional application No. 61/557,083, filed on Nov. 8, 2011.

(51) Int. Cl. A61N 1/05

(52) U.S. Cl. CPC A61N 1/05
USPC 607/116

(57) ABSTRACT

Apparatus and methods are described including an electrode device (30) that is configured to assume a coiled configuration when the device is in an unconstrained state, the electrode device being shaped to define a lumen (40). At least one electrode (21) is disposed on the electrode device. At least one electrical wire (32) is coupled to the electrode. A flexible lead (34) defines a lumen (42) therethrough, the electrical wire being housed inside the lead. A stylet (36) constrains the electrode device into a straightened configuration by being inserted into the lumen defined by the electrode device. The stylet additionally stiffens the flexible lead by being inserted into the lumen defined by the flexible lead. Other applications are also described.

### CROSS-REFERENCES TO RELATED APPLICATIONS

The present application claims the benefit of U.S. Provisional Patent Application 61/557,083 to Dagan, filed Nov. 8, 2011, entitled “Acute myocardial infarction treatment by electrical stimulation of the thoracic aorta.”

The present application is related to U.S. Ser. No. 13/210,778 to Dagan, filed Aug. 16, 2011 (published as US 2012/0035679), entitled, “Acute myocardial infarction treatment by electrical stimulation of the thoracic aorta,” which is a continuation-in part of U.S. Ser. No. 12/957,799 to Gross (published as US 2011/0137370), filed Dec. 1, 2010, entitled “Thoracic aorta and vagus nerve stimulation,” which is a continuation-in-part of U.S. Ser. No. 12/792,227 to Gross (published as US 2010/0305392), filed Jun. 2, 2010, entitled “Thoracic aorta and vagus nerve stimulation,” which claims the benefit of (a) U.S. Provisional Patent Application 61/183,319 to Reisner, filed Jun. 2, 2009, entitled “Thoracic aorta and vagus nerve stimulation,” and (b) U.S. Provisional Patent Application 61/331,453 to Dagan, filed May 5, 2010, entitled “Thoracic aorta and vagus nerve stimulation.”

All of the above-referenced applications are incorporated herein by reference.

### FIELD OF EMBODIMENTS OF THE INVENTION

Some applications of the present invention generally relate to medical apparatus. Specifically, some applications of the present invention relate to apparatus and methods for treatment of acute myocardial infarction.

### BACKGROUND

Acute myocardial infarction (AMI) is the result of interruption of blood supply to a part of the heart, causing heart cells to die. During AMI, damage is caused to the cardiac tissue by prolonged ischemia as well as due to injury during reperfusion.

Neurohormonal modulation, including inhibition of the sympathetic tone and activation of the parasympathetic tone to the heart, has been shown to have a protective effect on the cardiac tissue during ischemia and during reperfusion of the heart.

Beta blockers, which inhibit the beta sympathetic tone, are typically used in the treatment of AMI. There is evidence that intravenous beta blockers, administered acutely to treat AMI, reduce in-hospital mortality resulting from myocardial infarction, and are also useful in the control of the pain associated with AMI. Acute intravenous administration of beta blockers has been shown to improve the myocardial oxygen supply-demand relationship, decrease pain, reduce infarct size, and decrease the incidence of serious ventricular arrhythmias.

Typically, percutaneous coronary interventions are used to treat AMI. For example, procedures such as balloon dilatation and stent implantation are used to open a stenosis in the coronary arteries so as to re-perfuse the heart.

Reperfusion therapy is typically used in the treatment of ST elevation myocardial infarction (STEMI). Primary percutaneous coronary intervention (PCI) typically improves survival of STEMI. Ischemia and/or reperfusion injury is still an unresolved problem in STEMI treated by reperfusion therapy. Primary PCI can restore epicardial flow in 90-95% of patients, but 15-20% of patients do not achieve microvascular flow. Lack of microvascular perfusion after STEMI is the primary determinant of LV remodeling.

### SUMMARY OF EMBODIMENTS

For some applications of the present invention, a subject suffering from AMI is identified. The subject is treated by percutaneously placing at least one electrode inside the subject's aorta in contact with an aortic site, and electrically stimulating the aortic site. The aortic site is typically between the bifurcation of the aorta with the left subclavian artery and the bifurcation of the aorta with the fourth intercostal artery.

Typically, a plurality of electrodes are placed in contact with the aortic site. For some applications, the electrodes are disposed on an intra-aortic electrode device. The intra-aortic electrode device is shaped to have a coiled configuration, when the device is unconstrained. The intra-aortic electrode device is configured to be deployed at the aortic site by the device expanding against the inner wall of the aorta, by the device assuming a coiled configuration inside the aorta. The intra-aortic electrode device is thereby configured to maintain the position of the electrodes with respect to the aortic site. The electrodes are typically coupled to an external control unit, via electrical wires, which are housed in a flexible lead that passes from the electrode device to outside the subject's body.

Typically, the intra-aortic electrode device, and the lead that houses the electrical wires define lumens. During advancement of the intra-aortic electrode device and the lead toward the aortic site, a stylet is placed inside the lumens of the intra-aortic electrode device and the lead. Typically, the stylet stiffens the intra-aortic electrode device and the lead, such as to facilitate the advancement of the intra-aortic electrode device and the lead toward the aortic site. Further typically, insertion of the stylet into the lumen defined by the intra-aortic electrode device constrains the device into a straightened configuration.

When the intra-aortic electrode device is disposed in a vicinity of the aortic site, the stylet is gradually removed from the lumen defined by the intra-aortic electrode device, causing the intra-aortic electrode device to expand against the inner wall of the aorta by assuming a coiled configuration, thereby maintaining the position of the electrodes with respect to the aortic site. Subsequently, the stylet is removed from the lumen defined by the lead, such that the lead becomes flexible. Typically, the flexibility of the lead is such that the lead can accommodate movement of the subject, such that the subject's movement does not cause the intra-aortic electrode device to become displaced.

The electrical stimulation of the aortic site typically reduces afterload by suppressing the sympathetic tone of the heart and vasculature. Further typically, the stimulation of the aortic site reduces cardiac oxygen consumption, left ventricular workload, and/or coronary microvascular constriction, and/or induces cardiac microvascular dilation. For some applications, the electrical stimulation reduces the likelihood of a lethal arrhythmia occurring.

For some applications, total peripheral resistance of the subject is reduced, and/or aortic compliance is increased by applying the electrical stimulation. For some applications, the electrical stimulation causes a reduction in heart rate, left ventricular pressure, left ventricular oxygen consumption, left ventricular wall stress, and/or left ventricular external work. For some applications, one or more of the aforementioned effects are caused by the electrical stimulation of the aortic site activating afferent signals traveling via the left vagus nerve. For some applications, one or more of the aforementioned effects are achieved by the electrical stimulation of the aortic site suppressing the sympathetic tone and/or increasing the parasympathetic tone of the heart and vasculature.

Typically, the electrical stimulation is applied during and/or subsequent to the performance of a percutaneous coronary intervention on the subject. For example, the electrical stimulation may be applied during and/or subsequent to (e.g., immediately subsequent to) balloon dilatation, and/or stent implantation, in order to open a stenosis in the coronary arteries so as to re-perfuse the subject's heart. The electrical stimulation reduces afterload (in conjunction with causing additional effects, as described hereinabove), during and/or subsequent to the intervention. For some applications, the electrical stimulation is applied for a period of time subsequent to the intervention having been performed, e.g., so as to protect the cardiac tissue by reducing afterload, cardiac oxygen consumption, left ventricular workload, and/or coronary microvascular constriction, and/or by inducing cardiac microvascular dilation, and/or by reducing reperfusion injury and apoptosis (in conjunction with causing additional effects, as described hereinabove), during reperfusion of the heart, subsequent to the intervention.

There is therefore provided, in accordance with some applications of the present invention, apparatus, including:

an electrode device that is configured to assume a coiled configuration when the device is in an unconstrained state, the electrode device being shaped to define a lumen;

at least one electrode disposed on the electrode device;

at least one electrical wire coupled to the electrode;

a flexible lead that defines a lumen therethrough, the electrical wire being housed inside the lead; and

a stylet configured to:
constrain the electrode device into a straightened configuration by being inserted into the lumen defined by the electrode device, and
stiffen the flexible lead by being inserted into the lumen defined by the flexible lead.

For some applications, the electrode device and the flexible lead include respective portions of a single integrated structure.

For some applications, a length of the flexible lead is less than 130 cm.

For some applications, the length of the flexible lead is less than 110 cm.

For some applications, a length of the flexible lead is more than 60 cm.

For some applications, the length of the flexible lead is more than 80 cm.

For some applications, the electrode device includes distal and proximal portions thereof, the at least one electrode is disposed on the distal portion of the electrode device, and no electrodes are disposed on the proximal portion of the electrode device.

For some applications, the at least one electrode includes a plurality of electrodes that are disposed on the distal portion of the electrode device, and in the unconstrained configuration of the electrode device, the distal portion is configured to define a substantially circular shape, and a distance between any one of the electrodes and an adjacent one of the electrodes is approximately equal for all of the electrodes disposed on the distal portion of the electrode device.

For some applications, a flexibility of the distal portion of the electrode device is less than a flexibility of the proximal portion of the electrode device.

For some applications, in the unconstrained configuration of the electrode device, the distal portion has a substantially circular shape and the proximal portion has a helical shape.

For some applications, the electrode device is configured such that in the unconstrained state of the electrode device, the device defines a coil having an outer diameter that is less than 40 mm.

For some applications, the electrode device is configured such that in the unconstrained state of the electrode device, the device defines a coil having an outer diameter that is less than 35 mm.

For some applications, the electrode device is configured such that in the unconstrained state of the electrode device, the device defines a coil having an outer diameter that is more than 15 mm.

For some applications, the electrode device is configured such that in the unconstrained state of the electrode device, the device defines a coil having an outer diameter that it more than 20 mm.

For some applications, the electrode device is configured such that in the unconstrained state of the electrode device, the device has a length of less than 80 mm.

For some applications, the electrode device is configured such that in the unconstrained state of the electrode device, the device has a length of less than 70 mm.

For some applications, the electrode device is configured such that in the unconstrained state of the electrode device, the device has a length of more than 30 mm.

For some applications, the electrode device is configured such that in the unconstrained state of the electrode device, the device has a length of more than 40 mm.

For some applications, the electrode device is configured such that in the straightened configuration of the electrode device, the device has a length of less than 400 mm.

For some applications, the electrode device is configured such that in the straightened configuration of the electrode device, the device has a length of less than 300 mm.

For some applications, the electrode device is configured such that in the straightened configuration of the electrode device, the device has a length of more than 100 mm.

For some applications, the electrode device is configured such that in the straightened configuration of the electrode device, the device has a length of more than 150 mm.

There is further provided, in accordance with some applications of the present invention, a method, including:

percutaneously advancing toward a subject's aorta:
an electrode device that is shaped to define a lumen, the electrode device having at least one electrode disposed thereon, and
a flexible lead that is shaped to define a lumen therethrough, the flexible lead housing at least one electrical wire that is coupled to the electrode,
the advancing being performed while a stylet is disposed inside the lumens defined by the electrode device and the flexible lead, such as to stiffen the lead and the electrode device; and

when the electrode device is disposed inside the subject's aorta:
causing the electrode device to expand against an inner wall of the aorta, by the electrode device assuming a coiled configuration, by retracting the stylet from the lumen defined by the electrode device; and
causing the lead to become flexible by removing the stylet from the lumen defined by the lead.

For some applications, advancing the electrode device and the lead toward the aorta includes advancing the electrode device and the lead toward the aorta, the electrode device and the flexible lead including respective portions of a single integrated structure.

For some applications, the method further includes, subsequent to retracting the stylet from the lumen defined by the electrode device, causing the electrode device to straighten from the coiled configuration of the electrode device by re-inserting the stylet into the lumen defined by the electrode device.

For some applications, causing the electrode device to expand against an inner wall of the aorta, by the electrode device assuming a coiled configuration, includes causing a distal portion of the electrode device to assume substantially circular shape, and causing a proximal portion of the electrode device to assume a substantially helical shape.

For some applications, the electrode device includes an electrode device having a plurality of electrodes disposed on the distal portion of the device, and no electrodes disposed on the proximal portion of the electrode device, and causing the distal portion of the electrode device to assume the substantially circular shape includes causing the distal portion to assume a shape that is such that a distance between any one of the electrodes and an adjacent one of the electrodes is approximately equal for all of the electrodes disposed on the distal portion of the electrode device

There is additionally provided, in accordance with some applications of the present invention, apparatus, including:

an electrode device that is shaped to define a lumen, the electrode device being configured such that in an unconstrained configuration of the electrode device, the electrode device defines:
a distal portion having a substantially circular shape, and
a proximal portion having a helical shape;

at least one electrode disposed on the distal portion of the electrode device; and

a stylet configured to:
constrain the electrode device into a straightened configuration by being inserted into the lumen defined by the electrode device.

For some applications, the at least one electrode includes a plurality of electrodes that are disposed on the distal portion of the electrode device, and a distance between any one of the electrodes and an adjacent one of the electrodes is approximately equal for all of the electrodes disposed on the distal portion of the electrode device.

For some applications, a flexibility of the distal portion of the electrode device is less than a flexibility of the proximal portion of the electrode device.

The present invention will be more fully understood from the following detailed description of embodiments thereof, taken together with the drawings, in which:

### BRIEF DESCRIPTION OF THE DRAWINGS

FIG. 1A is a schematic illustration of apparatus for acute treatment of a subject suffering from AMI, in accordance with some applications of the present invention;

FIG. 1B is a schematic illustration of an aortic site that is electrically stimulated, in accordance with some applications of the present invention;

FIGS. 2A-C are graphs showing experimental data that were obtained, in experiments conducted in accordance with some applications of the present invention;

FIG. 3 is a schematic illustration of an intra-aortic electrode device and a lead that houses electrical wires, in accordance with some applications of the present invention;

FIG. 4 is a schematic cross-sectional view of the lead that houses the electrical wires, in accordance with some applications of the present invention;

FIGS. 5-6 are schematic illustrations of the intra-aortic electrode device, in accordance with some applications of the present invention; and

FIGS. 7-8 are schematic illustrations of the intra-aortic electrode device, in accordance with some applications of the present invention.

### DETAILED DESCRIPTION OF EMBODIMENTS

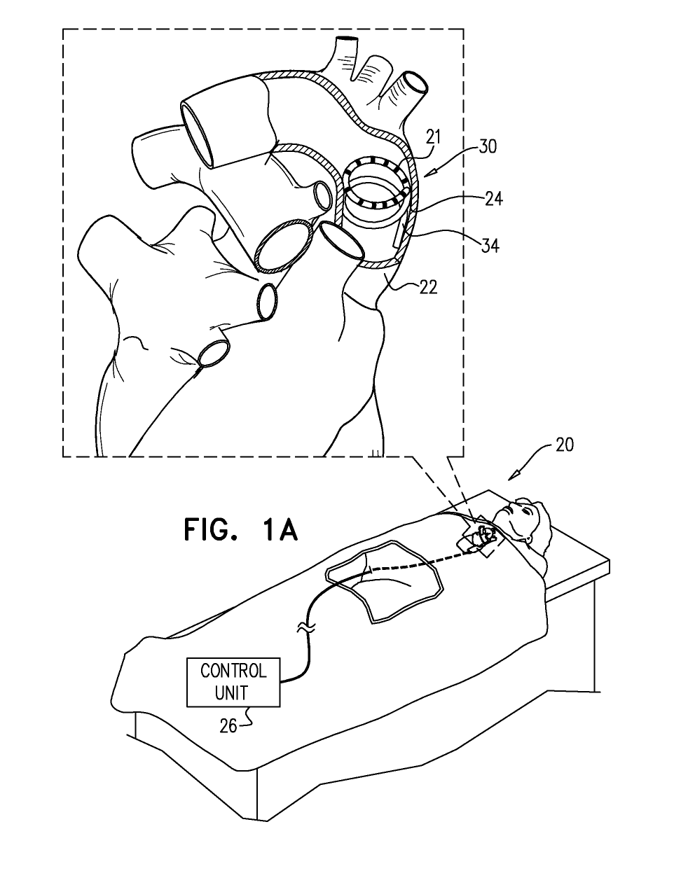

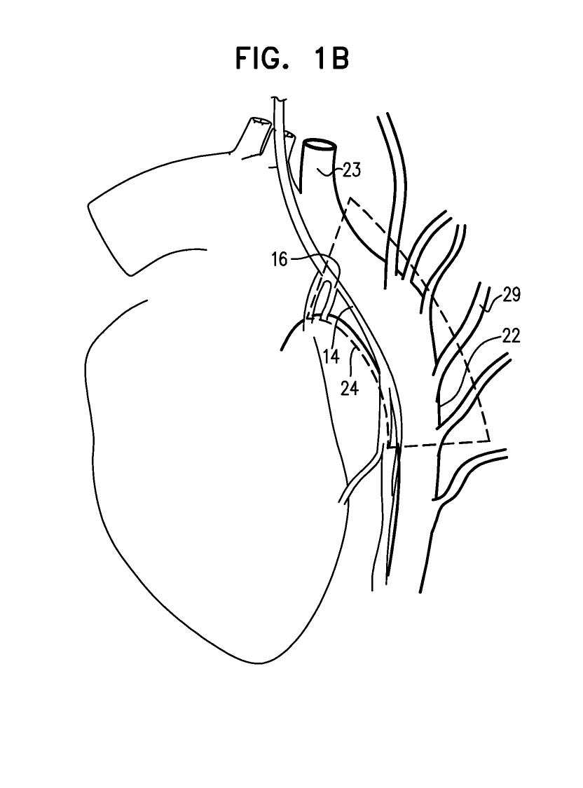

Reference is now made to FIGS. 1A-B, which are schematic illustrations of, respectively, apparatus 20 for treatment of a subject suffering from AMI, and an aortic site 24, in accordance with some applications of the present invention. For some applications of the invention, a subject suffering from AMI is identified. The subject is treated by percutaneously (e.g., transfemorally) placing at least one electrode 21 (typically, a plurality of electrodes) inside the subject's aorta 22 in contact with aortic site 24, and electrically stimulating the aortic site, by driving a current into the aortic site. The current is typically driven into the aortic site by a control unit 26 (e.g., a bedside work station) disposed outside the subject's body.

The electrical stimulation of the aortic site typically reduces afterload by suppressing the sympathetic tone, and/or increasing parasympathetic tone. Further typically, the stimulation of the aortic site reduces cardiac oxygen consumption, left ventricular workload, and/or coronary microvascular constriction, and/or induces cardiac microvascular dilation. For some applications, the electrical stimulation reduces the likelihood of a lethal arrhythmia occurring. Typically, electrical stimulation of the aortic site leads to prevention of ventricular remodeling.

For some applications, total peripheral resistance of the subject is reduced, and/or aortic compliance is increased by applying the electrical stimulation. For some applications, during reperfusion of the heart, subsequent to the infarction, application of the electrical stimulation increases microvascular perfusion by causing vasodilation of the coronary arterioles, and/or protects ventricular myocytes from reperfusion injury (e.g., apoptosis and/or necrosis). For some applications, the electrical stimulation causes a reduction in heart rate, left ventricular pressure, aortic pressure, left ventricular oxygen consumption, left ventricular wall stress and/or left ventricular external work. For some applications, one or more of the aforementioned effects are caused by the electrical stimulation of the aortic site activating afferent aortic signals traveling via the left vagus nerve. For some applications, one or more of the aforementioned effects are achieved by the electrical stimulation of the aortic site suppressing the sympathetic tone and/or increasing the parasympathetic tone of the heart and vasculature.

Typically, the percutaneously-inserted electrode is placed in contact with an aortic site 24 that is between the bifurcation of aorta 22 with the left subclavian artery 23 and the bifurcation of the aorta with the fourth intercostal artery 29, as shown in FIG. 1B. For some applications, the aortic site is adjacent to a portion of a vagus nerve 14 of the subject that is between (a) a vagal bifurcation 16 with a thoracic cardiac branch of the subject (i.e., the thoracic cardiac branch from the left recurrent laryngeal), and (b) thoracic vagal branching into the esophageal plexus of the subject. For some applications, the aortic site is adjacent to a portion of vagus nerve 14 that is slightly proximal to bifurcation 16, e.g., a portion of the vagus nerve between (a) the upper junction of the left thoracic vagal trunk with the left subclavian artery, and (b) bifurcation 16, such as a proximal thoracic location.

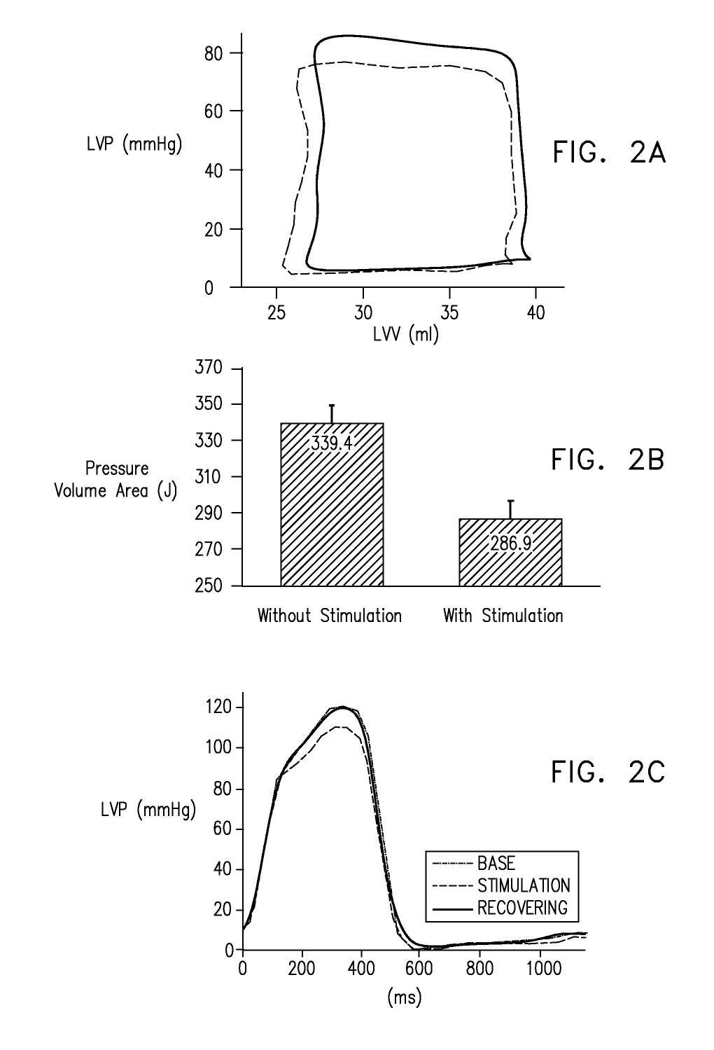

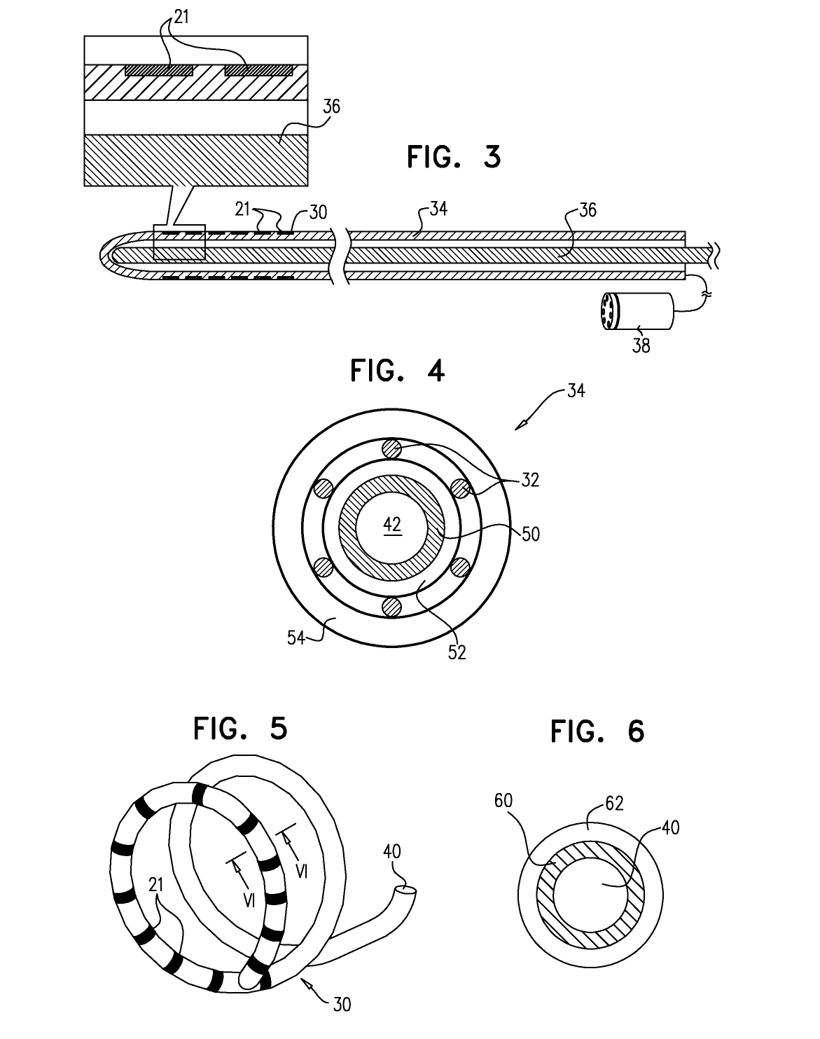

Typically, a plurality of electrodes 21 (e.g., two to ten electrodes) are disposed on a coiled intra-aortic electrode device 30, device 30 being as described hereinbelow with reference to FIG. 3 and FIGS. 5-8. Electrical wires 32 (FIG. 4), which are typically housed in a flexible lead 34 (FIG. 3), electrically couple the electrodes to external control unit 26. Typically, flexible lead 34 and electrode device 30 comprise two portions of a single integrated structure.

For some applications, subsequent to placing intra-aortic electrode device 30 into the subject's aorta, electrode 21 is successively placed in contact with respective locations of the inner wall of the aorta, and the respective locations of the wall of the aorta are electrically stimulated via the electrode. Changes in physiological parameters of the subject resulting from the stimulation of the respective locations are measured. Responsively thereto, one of the locations is selected as the aortic site, and the electrode is placed in contact with the selected location for the remainder of the treatment or for a portion thereof.

For some applications, subsequent to electrode 21 being placed at aortic site 24, and commencement of the electrical stimulation via the electrode, physiological parameters of the subject, such as heart rate, and/or blood pressure, are measured. Responsively thereto, parameters of the electrical stimulation that is applied to the aortic site are adjusted.

For some applications, physiological parameters of the subject are measured by detecting an electrical signal at the aortic site, for example via the at least one electrode 21, and/or via a different set of electrodes (not shown), the electrical signal being interpreted as being indicative of a physiological parameter of the subject. For some applications, the subject's cardiac cycle is determined by deriving the subject's ECG from the electrical signal detected at the aorta, and the electrical stimulation is applied to the aortic site in coordination with the subject's cardiac cycle.

Typically, the electrical stimulation is applied (at least periodically) while a percutaneous coronary intervention is performed on the subject. For example, the electrical stimulation may be applied while balloon dilatation, and/or stent implantation are performed, in order to open a stenosis in the coronary arteries so as to re-perfuse the subject's heart. The electrical stimulation reduces afterload, cardiac oxygen consumption, left ventricular workload, and coronary microvascular constriction, and/or induces cardiac microvascular dilation (in conjunction with causing additional effects, as described hereinabove), while the intervention is performed. Typically, intra-aortic electrode device 30 is inserted into the aorta via the same access point (typically, a femoral access point) as is used for the percutaneous coronary intervention.

For some applications, the electrical stimulation is applied for a period of time subsequent to the intervention having been performed, e.g., so as to protect the cardiac tissue by reducing afterload, cardiac oxygen consumption, left ventricular workload, and/or coronary microvascular constriction, and/or by inducing cardiac microvascular dilation, and/or by reducing reperfusion injury and apoptosis (in conjunction with causing additional effects, as described hereinabove), during reperfusion of the heart, subsequent to the intervention. Typically, the electrical stimulation is initiated during the intervention, and the stimulation continues for more than 1 hour and/or less than 72 hours (e.g., less than 24 hours). Subsequent to the termination of the electrical stimulation, lead 34 and electrode device 30 are removed from the subject's body (typically, immediately).

For some applications, the current is driven into the aortic site in coordination with the subject's cardiac cycle and/or respiratory cycle. For example, the subject's ECG may be detected, and the current may be driven into the electrode implantation site responsively to the detection of the QRS complex. Alternatively or additionally, the subject's blood pressure may be measured and the current may be driven responsively thereto. For some applications, the subject's ECG, and/or the subject's blood pressure is derived from an electrical signal detected at the aorta, using electrodes 21, or a different set of electrodes (not shown). Alternatively, the current is driven independently of the subject's cardiac cycle and/or respiratory cycle.

For some applications, driving current into aortic site 24, via electrodes 21, dilates the aorta by increasing nitric oxide (NO) secretion by the wall of the aorta, and/or by increasing the secretion of another vasodilation mediator from the wall of the aorta. Typically, driving current into aortic site 24, via electrodes 21, inhibits the sympathetic system tone and enhances parasympathetic tone by activation of aortic afferent fibers.

For some applications, driving current into aortic site 24, via electrode 21, activates afferent aortic signals traveling via the left vagus nerve thereby stimulating autonomic control centers in the central nervous system such as to enhance parasympathetic tone, thereby eliciting a parasympathetic response. For some applications, driving current into the aortic site generates an aortic response, as described hereinabove, in addition to generating the aforementioned vagal response. For some applications, driving the current into the aortic site stimulates autonomic control centers in the central nervous system, thereby inhibiting sympathetic tone, and inhibiting sympathetic signaling to the heart and periphery.

For some applications, driving current into the aortic site, via electrode 21 reduces a ratio of a low frequency component (e.g., less than 0.05 Hz) to a high frequency component (e.g., 0.15-0.35 Hz) of heart rate variability of the subject. For some applications, driving current into the aortic site, via electrode 21 reduces a ratio of a low frequency component (e.g., less than 0.05 Hz) to a high frequency component (e.g., 0.15-0.35 Hz) of blood pressure variability of the subject.

For some applications, the current has a frequency of between 5 Hz and 150 Hz, e.g., more than 100 Hz and/or less than 150 Hz. For some applications, the current has an amplitude of between 1 mA and 15 mA, e.g., between 2 mA and 3 mA. For some applications, a current having two pulses to 40 pulses, e.g., five pulses to thirty pulses (such as 20-30 pulses), per cardiac cycle, is driven into the aorta. In accordance with respective applications, the current is delivered continuously or intermittently. The current may thus be applied, for example: (a) as an endless train of pulses, or (b) during scheduled non-contiguous stimulation periods.

In a typical application, the current is driven as a symmetric rectangular biphasic pulse with 2 ms positive current and 2 ms negative current, at a frequency of approximately 125 Hz. Typically, the pulses are driven in coordination with the subject's QRS complex, and more than twenty pulses and/or less than forty pulses (e.g., approximately thirty pulses) are driven per cardiac cycle. Further typically, the pulses, cyclically, are driven into the aortic site during a stimulation period, and are not driven into the aortic site during rest periods between consecutive stimulation periods. For some applications, each stimulation period is more than 1 minute and/or less than three minutes, e.g., about two minutes. For some applications, each rest period is more than two minutes and/or less than four minutes, e.g., about three minutes.

Reference is now made to FIGS. 2A-C, which are graphs showing experimental data that were obtained in experiments conducted in accordance with some applications of the present invention.

The graph shown in FIG. 2A shows the left ventricular pressure versus left ventricular volume curve of a dog with induced heart failure. The left ventricular pressure and left ventricular volume of the dog were measured using a Millar pressure-volume (P-V) conductance catheter system, in accordance with techniques described in Angeli et al., Cardiovasc Drugs Ther (2010) 24:409-420. The solid curve in the graph shown in FIG. 2A is the dog's left ventricular pressure versus left ventricular volume curve as measured over a period of two to three minutes, before the dog's aorta was electrically stimulated. The dog's aorta was electrically stimulated at an aortic site between the bifurcation of the aorta with the left subclavian artery and the bifurcation of the aorta with the first intercostal artery. The dog was stimulated acutely using the LASSO 2515 Variable Circular Mapping Catheter with 10 mA, 125 Hz, symmetric biphasic rectangular pulses with 2 ms positive current and 2 ms negative current.

The dashed curve shown in FIG. 2A is the dog's left ventricular pressure versus left ventricular volume curve as measured while the dog was being stimulated for a period of two minutes. Subsequent to the termination of the electrical stimulation at the aortic site, the dog's left ventricular pressure versus volume curve returned to the pre-stimulation curve, i.e., the solid curve shown in FIG. 2A.

A shift in a left ventricular pressure versus left ventricular volume curve toward the x and y axes (i.e., downward and to the left) is indicative of afterload reduction, as described in David Kass, Eur Heart J. 1992 November; 13 Suppl E:57-64, which is incorporated herein by reference. Thus, the shift of the left ventricular pressure versus volume curve of the dog downward and to the left during the electrical stimulation of the aortic site is indicative of the fact that the acute electrical stimulation of the dog's aorta resulted in a reduction in afterload during the stimulation period. These data indicate that acutely stimulating an aortic site in accordance with techniques described herein, will cause a reduction in a subject's afterload at least during the stimulation period. Thus, in accordance with some applications of the present invention, the aortic site of a subject suffering from a myocardial infarction is stimulated during, and/or for a period subsequent to, a percutaneous coronary intervention being applied to the subject, so as to reduce the subject's afterload during the application of the stimulation. Alternatively or additionally, the aortic site of a subject suffering from a different condition is stimulated acutely, so as to reduce the subject's afterload during the application of the stimulation.

The bar chart shown in FIG. 2B shows the mean left ventricular pressure-volume area of a group of six dogs. All of the dogs were suffering from heart failure, and had had electrodes implanted at an aortic site between the bifurcation of the aorta with the left subclavian artery and the bifurcation of the aorta with the first intercostal artery. A Millar pressure-volume (P-V) conductance catheter system was placed in the left ventricle of each of the dogs to facilitate measurement of left ventricular pressure and left ventricular volume of the dogs. An inflatable balloon catheter was placed in the inferior vena cava of each of the dogs.

Baseline PVA curves for the dogs were recorded, while electrical stimulation was not being applied to the aortic sites of the dogs. The baseline curves were recorded for each of the dogs by inflating the inferior-vena-cava balloon for several seconds and then deflating the balloon. The left ventricular pressure and volume of the dogs was measured during the inflation and the subsequent deflation, using the Millar pressure-volume (P-V) conductance catheter system, in accordance with techniques described in Steendijk et al., European Heart Journal (2004) 6 (Supplement D), D35-D42, which is incorporated herein by reference. The average pressure volume area of the dogs’ baseline PVA curves was determined and is plotted as the left bar of the bar chart shown in FIG. 2B.

Subsequently, electrical stimulation of the aortic sites of the dogs was initiated, and it was determined that, as a result of the electrical stimulation, the dogs had undergone a blood pressure decrease that had stabilized. Subsequent to the stabilization of the blood pressure decrease, stimulation PVA curves for the dogs were recorded, while the electrical stimulation continued to be applied to the aortic sites of the dogs. The stimulation curves were recorded for each of the dogs by inflating the inferior-vena-cava balloon for several seconds and then deflating the balloon. The left ventricular pressure and volume of the dog was measured during the inflation and the subsequent deflation, using the Millar pressure-volume (P-V) conductance catheter system. The average pressure volume area of the dogs' stimulation PVA curves was determined and is plotted as the right bar of the bar chart shown in FIG. 2B.

There is a highly linear correlation between the pressure volume area of a subject's left ventricle and myocardial oxygen consumption per heartbeat, as described in Suga et al., Am J Physiol. 1981 January; 240(1):H39-44, which is incorporated herein by reference. This relationship holds true under a variety of loading and contractile conditions. This estimation of myocardial oxygen consumption is used to study the coupling of mechanical work and the energy requirement of the heart in various disease states, such as diabetes, ventricular hypertrophy and heart failure. Myocardial oxygen consumption is also used in the calculation of cardiac efficiency, which is the ratio of cardiac stroke work to myocardial oxygen consumption. As shown in FIG. 2B, acute stimulation of the aortic site of the heart failure dogs using techniques described herein substantially reduced the left ventricular pressure volume area of the dogs relative to the baseline pressure volume area of the dogs. The data had a P-value of 0.05. The data shown in FIG. 2B indicate that acutely stimulating an aortic site, in accordance with techniques described herein, will cause a reduction in a subject's myocardial oxygen consumption at least during the stimulation period. Thus, in accordance with some applications of the present invention, the aortic site of a subject suffering from a myocardial infarction is stimulated during, and/or for a period subsequent to, a percutaneous coronary intervention being applied to the subject, so as to reduce the subject's myocardial oxygen consumption during the application of the stimulation. Alternatively or additionally, the aortic site of a subject suffering from a different condition is stimulated acutely, so as to reduce the subject's myocardial oxygen consumption during the application of the stimulation.

FIG. 2C is a graph showing left ventricular pressure as measured in a post-myocardial infarction human subject that generally suffered from heart failure. The subject was treated by acutely stimulating an aortic site of the subject between the bifurcation of the aorta with the left subclavian artery and a location 4 cm downstream of the bifurcation. Electrical stimulation was applied to the aortic site for a stimulation period of two minutes, via electrodes that were disposed on a LASSO 2515 Variable Circular Mapping Catheter, manufactured by Biosense Webster. The subject's mean left ventricular pressure waveform over the course of a cardiac cycle is shown in FIG. 2C, as measured before stimulation (base), during stimulation, and two minutes subsequent to the stimulation (recovering). As shown in FIG. 2C, during application of the stimulation, the subject's left ventricular pressure was reduced. The subject's left ventricular pressure waveform returned to having a generally similar shape to the baseline shape subsequent to the termination of the electrical stimulation. These data indicate that acutely stimulating an aortic site in accordance with techniques described herein, will cause a reduction in a subject's left ventricular pressure at least during the stimulation period. Thus, in accordance with some applications of the present invention, the aortic site of a subject suffering from a myocardial infarction is stimulated during, and/or for a period subsequent to, a percutaneous coronary intervention being applied to the subject, so as to reduce the subject's left ventricular pressure during the application of the stimulation. Alternatively or additionally, the aortic site of a subject suffering from a different condition is stimulated acutely, so as to reduce the subject's left ventricular pressure during the application of the stimulation.

Reference is now made to FIGS. 3-6, which are schematic illustrations of intra-aortic electrode device 30, and lead 34, which houses electrical wires 32, in accordance with some applications of the present invention. FIG. 3 shows intra-aortic electrode device 30 and lead 34 with a stylet 36 disposed therein, in accordance with some applications of the present invention. FIG. 4 shows a cross-sectional view of lead 34. FIG. 5 shows intra-aortic electrode device 30 in an unconstrained configuration, and FIG. 6 shows a cross-sectional view of a portion of the intra-aortic electrode device.

For some applications, electrodes 21 are disposed on an intra-aortic electrode device 30. The intra-aortic electrode device is shaped to have a coiled configuration, when the device is unconstrained, as shown in FIG. 5, which shows device 30 in an unconstrained state. The intra-aortic electrode device is configured to be deployed at the aortic site by the device expanding against the inner wall of the aorta, by the device assuming a coiled configuration inside the aorta. The intra-aortic electrode device is thereby configured to maintain a position of the electrodes with respect to the aortic site. The electrodes are typically coupled to an external control unit, via electrical wires 32, which pass from the electrodes to outside the subject's body via flexible lead 34. For some applications, the electrical wires are coupled to an electrical connector 38, which in turn is configured to be coupled to external control unit 26 (FIG. 1A).

Typically, intra-aortic electrode device 30 defines a lumen 40 (FIG. 6), and lead 34 defines a lumen 42 (FIG. 4). During advancement of the intra-aortic electrode device and the lead toward the subject's aortic site, stylet 36 is placed inside the lumens of the intra-aortic electrode device and the lead, as shown in FIG. 3. Typically, the stylet stiffens the intra-aortic electrode device and the lead, such as to facilitate the advancement of the electrode device and the lead toward the aortic site. Further typically, insertion of the stylet into the lumen defined by the intra-aortic electrode device constrains the electrode device into a straightened configuration, as shown in FIG. 3. The intra-aortic electrode device and the lead are typically advanced toward the aortic site from a femoral access point, under fluoroscopic guidance.

When intra-aortic electrode device 30 is disposed in a vicinity of aortic site 24, stylet 36 is gradually retracted from lumen 40 defined by the intra-aortic electrode device, causing the intra-aortic electrode device to expand against the inner wall of the aorta by assuming a coiled configuration. The intra-aortic electrode device thereby maintains the position of electrodes 21 with respect to aortic site 24.

For some applications, subsequent to placing intra-aortic electrode device 30 into the subject's aorta, electrodes 21 are successively placed in contact with respective locations of the inner wall of the aorta, by expanding the electrode device at respective locations. The respective locations of the wall of the aorta are electrically stimulated via the electrodes. For example, the stylet may be removed from lumen 40 defined by device 30, when device 30 is disposed at a first location inside the aorta, and the aorta may be stimulated when the device is in the expanded state at the first location. Subsequently, (a) the stylet may be reinserted into lumen 40, (b) device 30 may be moved to a second location within the aorta, (c) the stylet may be again be removed from inside lumen 40, so as to facilitate expansion of device 30 at the second location, and (d) the aorta may be stimulated when the device is in the expanded state at the second location. Changes in physiological parameters of the subject resulting from the stimulation of the respective locations are measured. Responsively thereto, one of the locations is selected as the aortic site. Device 30 is deployed at the selected location for the remainder of the treatment (or for a portion thereof), by removing the stylet from lumen 40 of the device, while the device is disposed at the selected location.

Subsequent to the deployment of intra-aortic electrode device 30 at aortic site 24, stylet 36 is removed from lumen 42 defined by lead 34, such that the lead becomes flexible. Typically, the flexibility of the lead is such that the lead can accommodate movement of the subject, such that the subject's movement does not cause intra-aortic electrode device 30 to become displaced. Typically, subsequent to the removal of the stylet from lumen 42 of lead 34, lead 34 is advanced (e.g., advanced by several centimeters) into the access point, and a securing valve is then used to lock the lead in place with respect to the access point.

Typically, subsequent to treatment of the subject by the intra-aortic electrode device, stylet 36 is reintroduced into lumen 42 of lead 34, and into lumen 40 of device 30. The stylet stiffens device 30 and lead 34, thereby facilitating removal of device 30 and lead 34 from the subject's body.

It is noted that although lead 34 and electrode device 30 have been described as separate devices, typically the lead and the electrode device comprise two portions of a single integrated structure. Further typically, lumens 40 and 42 comprise distal and proximal portions of a single continuous lumen. Alternatively, for some applications, lead 34 and electrode device 30 do not form a single integrated structure.

Reference is again made to FIG. 4, which shows a cross-sectional view of lead 34. For some applications, lead 34 defines an inner, flexible metal coil 50. For example, the coil may include nitinol, and/or stainless steel. For some applications, lead 34 further defines inner and outer layers 52 and 54 of flexible tubing, e.g., flexible silicone, and/or PTFE tubing. Typically, electrical wires 32 are disposed between the inner and outer layers of flexible tubing, as shown in FIG. 4. For some applications, an anti-coagulation agent is disposed on the outer surface of outer tubing 54. Typically, the length of lead 34 is less than 130 cm (e.g., less than 110 cm), and/or more than 60 cm (e.g., more than 80 cm), e.g., 60-130 cm (e.g., 80-110 cm).

Reference is again made to FIG. 6, which shows a cross-sectional view of intra-aortic electrode device 30, in accordance with some applications of the present invention. For some applications, the device defines an inner tube 60 that includes a shape-memory alloy, such as nitinol. The inner tube is shape set to assume a coiled configuration when the inner tube is in an unconstrained state. Typically, the device defines an outer layer 62 that is an electrical insulator, e.g., silicone, or plastic. Electrodes 21 are disposed on the outer layer, the outer layer being configured to electrically isolate the electrodes.

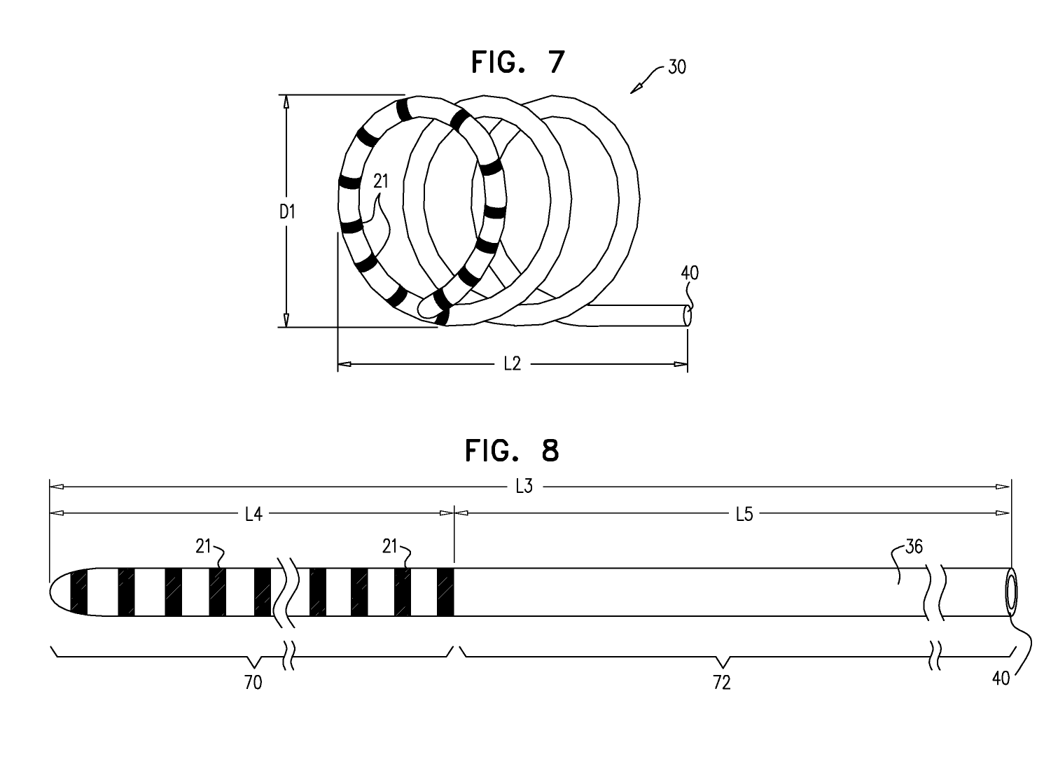

Reference is now made to FIGS. 7-8, which are schematic illustrations of intra-aortic electrode device 30, in accordance with some applications of the present invention. FIG. 7 shows device 30 in the unconstrained state of the device, and FIG. 8 shows device 30 as the device is shaped when the device is constrained into a straightened configuration by stylet 36 (not shown). Device 30, as shown in FIGS. 7-8, functions in a generally similar manner to that described hereinabove.

For some applications, electrodes 21 are disposed on a distal portion 70 of intra-aortic electrode device 30. A proximal portion 72 of device 30 functions as a support portion of the intra-aortic electrode device, portion 72 being configured to expand against the inner walls of the aorta such as to maintain a position of device 30 with respect to the aorta. For some applications, the proximal portion functions as a support portion that is configured maintain a position of device 30 with respect to the aorta, and not electrodes are disposed on the proximal portion, as shown.

For some applications, distal portion 70 is configured to assume a substantially circular shape (i.e., a planar circular shape) in an unconstrained configuration of intra-aortic electrode device 30. Electrodes are disposed along the length of the distal portion, such that in the unconstrained state of device 30, electrodes are disposed around the circumference of the distal portion of the device. For some applications, the electrodes are disposed along the length of the distal portion, such that in the unconstrained state of device 30, electrodes are disposed around the circumference of the distal portion of the device, such that a distance between any electrode and an adjacent electrode is approximately equal for all of the electrodes disposed around the circumference.

As described hereinabove, when device 30 is disposed in a vicinity of aortic site 24, stylet 36 is gradually retracted from lumen 40 defined by the intra-aortic electrode device, causing the intra-aortic electrode device to expand against the inner wall of the aorta. Typically, device 30 is configured to expand at an aortic site along the length of the aorta, such that, at the aortic site, electrodes 21 are placed in contact with the inner wall of the aorta in a spaced configuration around substantially the full circumference of the inner wall of the aorta. For some applications, device 30 is expanded such that, at the aortic site, a distance between any electrode and an adjacent electrode is approximately equal for all of the electrodes disposed around the circumference of the inner wall of the aorta. For some applications, since, at the aortic site, electrodes 21 are placed in contact with the inner wall of the aorta in a spaced configuration around substantially the full circumference of the inner wall of the aorta, device 30 does not need to be rotationally aligned with aortic site 24.

As described hereinabove, for some applications, changes in physiological parameters of the subject resulting from the stimulation of the respective locations within the aorta are measured. Responsively thereto, one of the locations is selected as the aortic site. Typically, at each longitudinal location along the aorta at which device 30 is expanded, electrodes 21 are placed in contact with the inner wall of the aorta in a spaced configuration around substantially the full circumference of the inner wall of the aorta. For some applications, when the device is expanded each given longitudinal location, changes in physiological parameters of the subject resulting from the stimulation the aorta at the longitudinal location using respective electrodes, or sets of electrodes, are measured. Responsively thereto, one of the electrodes, or sets of electrodes, is selected for stimulating the aorta at the longitudinal location. Typically, if at a given longitudinal location, none of the of the electrodes, or sets of electrodes, stimulates the aorta in a manner that causes a desired change in physiological parameters of the subject, then the electrode device is moved to a different longitudinal location, as described hereinabove.

Typically, proximal portion 72 of intra-aortic electrode device is configured to assume a helical shape in an unconstrained configuration thereof, as shown in FIG. 7. Thus, when stylet 36 is removed from lumen 40 of device 30 (as described hereinabove), proximal portion 72 expands against the inner wall of the aorta, in a helical shape, such as to support maintain a position of the intra-aortic electrode device with respect to aortic site 24 (i.e., such as to maintain distal portion 70 of device 30 at aortic site 24). Typically, the helical shape of the proximal portion of the electrode device is such as to facilitate blood flow through the aorta, since the proximal portion is disposed against the walls of the aorta and not in the center of the aorta. For some applications, lead 34 (FIG. 1A) is placed along the wall of the aorta, and not along the center of the aorta.

As described hereinabove, for some applications, device 30 defines an inner tube 60 that includes a shape-memory alloy, such as nitinol. Typically, device 30 is configured such that in the unconstrained configuration of the device, distal portion 70 assumes a substantially circular shape (i.e., a planar circular shape), and proximal portion assumes a helical shape, by pre-shaping of the shape-memory alloy. For some applications, distal portion 70 is configured to be less flexible than proximal portion 72, for example, by configuring the shape-memory alloy accordingly.

Typically, device 30 is configured such that in the unconstrained state of the device (FIG. 7), the device has a maximum outer diameter D1 of less than 40 mm (e.g., less than 35 mm), and/or more than 15 mm (e.g., more than 20 mm), such as 15-40 mm (e.g., 20-35 mm). Typically, device 30 is configured such that in the unconstrained state of the device, the device has a length L2 of less than 80 mm (e.g., less than 70 mm), and/or more than 30 mm (e.g., more than 40 mm), e.g., 30-80 mm (e.g., 40-70 mm).

For some applications, in the straightened configuration of device 30 (FIG. 8), total length L3 of device 30 is less than 400 mm (e.g., less than 300 mm), and/or more than 100 mm (e.g., more than 150 mm). For some applications, length L4 of distal portion 70 of device 30 is less than 130 mm (e.g., less than 110 mm), and/or more than 45 mm (e.g., more than 60 mm). For some applications, length L5 of proximal portion 72 of device 30 is less than 260 mm (e.g., less than 200 mm), and/or more than 30 mm (e.g., more than 60 mm). For some applications, a ratio of L5 to L4 is less than 3:1 (e.g., less than 2:1), and/or more than 0.5:1 (e.g., more than 1:1).

For some applications, the techniques described herein are practiced in combination with techniques described in PCT Publication WO 07/013065 to Gross, which is incorporated herein by reference. For some applications, the techniques described herein are practiced in combination with the techniques described in PCT application WO 09/095918, entitled “Peristaltic pump for treatment of erectile dysfunction,” to Gross, which claims priority from US Patent Application 2009/0198097 to Gross, the PCT application and the US application being incorporated herein by reference. For some applications, the techniques described herein are practiced in combination with the techniques described in US Patent Application 2009/0198097 to Gross, which is incorporated herein by reference. For some applications, the techniques described herein are practiced in combination with the techniques described in US 2011/0137370 to Gross, US 2010/0305392 to Gross, and/or US 2012/0035679 to Dagan, all of which applications, are incorporated herein by reference.

For some applications, the methods described herein are performed in combination with the techniques described in PCT Application WO 09/095920 to Gross, which is incorporated herein by reference.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. An apparatus, comprising:
an electrode device that comprises distal and proximal portions thereof, and that is shaped to define a lumen;
at least one electrode disposed on the distal portion of the electrode device, there being no electrodes disposed on the proximal portion of the electrode device,
the electrode device being configured, when in a non-constrained configuration thereof, to assume a coiled configuration, in which the distal portion has a substantially circular shape and the proximal portion has a helical shape;
at least one electrical wire coupled to the electrode;
a flexible lead that defines a lumen therethrough, the electrical wire being housed inside the lead; and
a stylet configured to:
constrain the electrode device into a straightened configuration by being inserted into the lumen defined by the electrode device, and
stiffen the flexible lead by being inserted into the lumen defined by the flexible lead.

2. The apparatus according to claim 1, wherein the electrode device and the flexible lead comprise respective portions of a single integrated structure.

3. The apparatus according to claim 1, wherein a length of the flexible lead is less than 130 cm.

4. (canceled)

5. The apparatus according to claim 1, wherein a length of the flexible lead is more than 60 cm.

6-7. (canceled)

8. The apparatus according to claim 1, wherein the at least one electrode comprises a plurality of electrodes that are disposed on the distal portion of the electrode device, and wherein in the unconstrained configuration of the electrode device, a distance between any one of the electrodes and an adjacent one of the electrodes is approximately equal for all of the electrodes disposed on the distal portion of the electrode device.

9. The apparatus according to claim 1, wherein a flexibility of the distal portion of the electrode device is less than a flexibility of the proximal portion of the electrode device.

10. (canceled)

11. The apparatus according to claim 1, wherein the electrode device is configured such that in the unconstrained state of the electrode device, the device defines a coil having an outer diameter that is less than 40 mm.

12. The apparatus according to claim 11, wherein the electrode device is configured such that in the unconstrained state of the electrode device, the device defines a coil having an outer diameter that is less than 35 mm.

13. The apparatus according to claim 1, wherein the electrode device is configured such that in the unconstrained state of the electrode device, the device defines a coil having an outer diameter that is more than 15 mm.

14. The apparatus according to claim 13, wherein the electrode device is configured such that in the unconstrained state of the electrode device, the device defines a coil having an outer diameter that is more than 20 mm.

15-16. (canceled)

17. The apparatus according to claim 1, wherein the electrode device is configured such that in the unconstrained state of the electrode device, the device has a length of more than 30 mm.

18. (canceled)

19. The apparatus according to claim 1, wherein the electrode device is configured such that in the straightened configuration of the electrode device, the device has a length of less than 400 mm.

20-22. (canceled)

23. A method, comprising:
percutaneously advancing toward a subject's aorta:
an electrode device that is shaped to define a lumen, the electrode device having at least one electrode disposed thereon, and
a flexible lead that is shaped to define a lumen therethrough, the flexible lead housing at least one electrical wire that is coupled to the electrode,
the advancing being performed while a stylet is disposed inside the lumens defined by the electrode device and the flexible lead, such as to stiffen the lead and the electrode device; and
when the electrode device is disposed inside the subject's aorta:
causing the electrode device to expand against an inner wall of the aorta, by the electrode device assuming a coiled configuration, by retracting the stylet from the lumen defined by the electrode device; and
causing the lead to become flexible by removing the stylet from the lumen defined by the lead.

24. The method according to claim 23, wherein advancing the electrode device and the lead toward the aorta comprises advancing the electrode device and the lead toward the aorta, the electrode device and the flexible lead comprising respective portions of a single integrated structure.

25. The method according to claim 23, the method further comprising, subsequent to retracting the stylet from the lumen defined by the electrode device, causing the electrode device to straighten from the coiled configuration of the electrode device by re-inserting the stylet into the lumen defined by the electrode device.

26. The method according to claim 23, wherein causing the electrode device to expand against an inner wall of the aorta, by the electrode device assuming a coiled configuration, comprises causing a distal portion of the electrode device to assume a substantially circular shape, and causing a proximal portion of the electrode device to assume a substantially helical shape.

27. The method according to claim 26, wherein the electrode device includes an electrode device having a plurality of electrodes disposed on the distal portion of the device, and no electrodes disposed on the proximal portion of the electrode device, and wherein causing the distal portion of the electrode device to assume the substantially circular shape comprises causing the distal portion to assume a shape that is such that a distance between any one of the electrodes and an adjacent one of the electrodes is approximately equal for all of the electrodes disposed on the distal portion of the electrode device

28. An apparatus, comprising:
an electrode device that is shaped to define a lumen, the electrode device being configured such that in an unconstrained configuration of the electrode device, the electrode device defines:
a distal portion having a substantially circular shape, and
a proximal portion having a helical shape;
at least one electrode disposed on the distal portion of the electrode device; and
a stylet configured to:
constrain the electrode device into a straightened configuration by being inserted into the lumen defined by the electrode device.

29. The apparatus according to claim 28, wherein the at least one electrode comprises a plurality of electrodes that are disposed on the distal portion of the electrode device, and wherein a distance between any one of the electrodes and an adjacent one of the electrodes is approximately equal for all of the electrodes disposed on the distal portion of the electrode device.

30. The apparatus according to claim 28, wherein a flexibility of the distal portion of the electrode device is less than a flexibility of the proximal portion of the electrode device.
